(19) United States
(12) Patent Application Publication
(10) Pub. No.: US 2002/0045271 A1
(43) Pub. Date: Apr. 18, 2002

(54) COMPOUNDS AND METHODS FOR IDENTIFYING COMPOUNDS THAT INTERACT WITH MICROSOMAL TRIGLYCERIDE TRANSFER PROTEIN BINDING SITES ON APOLIPOPROTEIN B AND MODULATE LIPID BIOSYNTHESIS

(75) Inventors: M. MAHMOOD HUSSAIN, ARDMORE, PA (US); AHMED BAKILLAH, PHILADELPHIA, PA (US)

Correspondence Address: JANE MASSEY LICATA, 66 E MAIN STREET, MARLTON, NJ, 08053

(73) Assignee: LICATA AND TYRRELL P.C.

(21) Appl. No.: 09/309,668

(22) Filed: May 11, 1999

Related U.S. Application Data
(60) Provisional application No. 60/088,767, filed on Jun. 10, 1998.

(51) Int. Cl. G01N 33/543

(52) U.S. Cl. 436/518

(57) ABSTRACT

Methods of identifying compositions which inhibit lipid and lipoprotein secretion are provided using in vitro assays measuring inhibition of the binding of microsomal triglyceride transfer protein to a specific microsomal triglyceride binding site on apolipoprotein B and lipid complexes.

This application claims benefit under 35 U.S.C. Section 119(e) from the United States provisional application, Serial No. 60/088,767, filed Jun. 10, 1998.

This invention was made in the course of research sponsored by the National Institutes of Health. The U.S. Government may have certain rights in this invention.

### BACKGROUND OF THE INVENTION

Apolipoprotein B (apoB), a translational product of the apoB gene, is an essential structural protein required for the assembly of triglyceride-rich lipoproteins. Transcription of the apoB gene occurs mainly in the small intestine and liver of adult animals. ApoB mRNAs are translated into single polypeptides of 2152 (apoB48) or 4536 (apoB100) amino acids in the intestine and liver, respectively. Unlike other secretory proteins, which are co-translationally inserted into the endoplasmic reticulum (ER) lumen, apoB is co-translationally integrated, at least transiently, into the ER membranes in a transmembrane orientation (Dixon, J. L. and H. N. Ginsberg 1993 J. Lipid Res. 34:167-179; Vance, J. E. and D. E. Vance 1990 Ann. Rev. Nutr. 10:337-356; Gibbons, G. F. 1990 Biochem. J. 268:1-13; Yao, Z. and R. S. McLeod 1994 Biochim. Biophys. Acta 1212:152-166; Sparks, J. D. and C .E. Sparks 1994 Biochim. Biophys. Acta 1215:9-32; Hussain, M. M. et al. 1996 Biochim. Biophys. Acta 1300:151-170; Innerarity, T. L. et al. 1996 J. Biol. Chem. 271:2353-2356). Translocation of apoB across the ER membrane is inefficient and probably determines lipoprotein production (Sakata, N. et al. 1993 J. Biol. Chem. 268:22967-22970; Bonnardel, J. A. and R. A. Davis 1995 J. Biol. Chem. 270:28892-28896). It is believed that apoB is lipidated even before completion of peptide synthesis as incompletely synthesized apoB polypeptides are secreted as lipoprotein particles by cells incubated with puromycin, a treatment which stops protein synthesis by releasing peptides from ribosomes (Boren, J. et al. 1992 J. Biol. Chem. 267: 9858-9867; Spring, D. J. et al. 1992 J. Biol. Chem. 267:14389-14845). Lipidation of apoB results in the release of apoB from the ER membrane and in the formation of primordial lipoproteins.

Microsomal triglyceride transfer protein (MTP) also plays an important role in lipoprotein assembly. The early lipidation of nascent apoB polypeptides requires MTP. Purified MTP activity consists of two subunits of 55 and 97 kDa. The 97 kDa subunit is essential for lipid transfer activity and is defective in abetalipoproteinemia patients who lack apoB-containing lipoproteins in their plasma (Sharp, D. et al. 1993 Nature 365:65-69; Wetterau, J. R. et al. 1992 Science 258:999-1001; Gregg, R. E. and J. R. Wetterau 1994 Curr. Opin. Lipidol. 5:81-86). The 55 kDa protein disulfide isomerase (PDI) subunit is required to keep the larger subunit in solution and to retain it in the ER (Wetterau, J. R. et al. 1991 Biochem. 30:9728-9735; Ricci, B. 1995 J. Biol. Chem. 270:14281-14285).

A direct correlation between MTP activity and lipoprotein assembly has been demonstrated in vitro by co-expressing apoB and MTP in cells that do not secrete lipoproteins (Gordon, D. A. et al. 1994 Proc. Natl. Acad. Sci. 91:7628-7632; Leiper, J. M. et al. 1994 J. Biol. Chem. 269:21951-21954; Gretch, D. G. et al. 1996 J. Biol. Chem. 271:8682-8691). In these studies, expression of apoB cDNAs resulted in the intracellular synthesis and degradation of apoB polypeptides, but no lipoprotein secretion. In contrast, co-transfection of these apoB-expressing cells with MTP resulted in the synthesis and secretion of apoB polypeptides (Gordon, D. A. et al. 1994 Proc. Natl. Acad. Sci. 91:7628-7632; Leiper, J.M . et al. 1994 J. Biol. Chem. 269:21951-21954; Gretch, D. G. et al. 1996 J. Biol. Chem. 271:8682-8691).

U.S. Pat. No. 5,595,872 and WO 96/40640 describe novel compounds identified as inhibitors of microsomal triglyceride transfer protein and/or apolipoprotein secretion which are believed to be useful in treating pancreatitis, obesity, hypercholesterolemia, hyper-triglyceridemia, hyperlipidemia, diabetes and atherosclerosis. The ability of these compounds to inhibit microsomal triglyceride transfer protein and/or apolipoprotein secretion was determined by measuring triglyceride secretion from human hepatoma cells in the presence and absence of compounds. Haghpassand et al., 1996 J. Lipid Research 37:1468-1480.

It has been suggested that MTP assists in the translocation of nascent apoB from the ER membrane (Gretch, D. G. et al. 1996 J. Biol. Chem. 271:8682-8691). Recently, evidence has been presented for a transient interaction between apoB and MTP in HepG2 cells using co-immunoprecipitations (Wu, X. J. et al. 1996 J. Biol. Chem. 271:10277-10281; Patel, S. B. and S. M. Grundy 1996 J. Biol. Chem. 271:18686-18694).

Assays for studying the interactions between MTP and apo-B-containing lipoproteins have been described by Bakillah et al. (Ninth Annual Mid-Atlantic Lipid Research Symposium, March 12-14, 1997). Using these assays, MTP was shown to interact with the N-terminus of apoB by ionic interactions which are modulated by phospholipids.

Little is known, however, concerning the biochemical, biophysical, or molecular nature of MTP/apoB interactions. Furthermore, those factors that play a role in the dissociation of MTP from nascent lipoproteins prior to secretion are not known.

MTP has been shown to exist in three different forms in the lumen of the endoplasmic reticulum; free, associated with lipids and bound to apo B. The specific binding site for MTP has now been identified and characterized. Further, the lipid associated MTP has been shown to bind to apoB with higher affinity. In addition, assays have been developed to study the association of MTP with lipid complexes. Accordingly, the present invention relates to methods of identifying compounds which inhibit the interaction of MTP or MTP/lipid complexes with apoB and the binding of MTP to lipid complexes. Compounds identified by these methods are useful in altering the secretion of lipids and lipoproteins.

### SUMMARY OF THE INVENTION

An object of the present invention is to provide a method of identifying compounds which modulate lipid secretion which comprises screening compounds in an assay which measures binding of MTP to a MTP specific binding site on apolipoprotein B. Compounds identified in the assay as inhibitors or inducers of binding of MTP to the MTP specific binding site on apoB should decrease or increase, respectively, lipid secretion.

Another object of the present invention is to provide a method of identifying compounds which modulate lipid secretion which comprises screening compounds in an assay which measures binding of MTP-lipid complexes to apoB. Compounds identified in the assay as inhibitors or inducers of binding of MTP-lipid complexes to apoB should decrease or increase, respectively, lipid secretion.

Yet another object of the present invention is to provide a method of identifying compounds that inhibit or increase the association of MTP with lipid complexes. These compounds may also modulate lipid secretion.

### DETAILED DESCRIPTION OF THE INVENTION

Atherosclerosis is associated with a variety of disorders including diabetes mellitus, hypertension, familial hypercholesterolemia, familial combined hyperlipidemia, familial dysbetalipoproteinemia, familial hypoalphalipo-proteinemia, hypothyroidism, cholesterol ester storage disease, systemic lupus erythematosus, and homocysteinemia. Much research into this disease has been focused on identifying risk factors common to those people affected by atherosclerosis. High plasma lipid and lipoprotein levels have been identified as risk factors for atherosclerosis. Accordingly, lowering plasma lipid levels has become a national goal and new approaches to decreasing lipid levels are required.

A microsomal triglyceride transfer protein (MTP) specific binding site on apoB has now been identified and characterized. In addition, methods have now been developed to study the association of MTP with lipid complexes. MTP binding to apoB is important as an early step in the lipidation of lipoproteins, which leads to increased secretion of these lipoproteins into the blood. Identifying agents that inhibit the binding of apoB and MTP will lead to decreasing apoB secretion. The separate step in synthesis of larger lipoproteins is believed to involve binding of MTP/lipid complexes to apoB. Therefore, inhibition of the binding of MTP/lipid complexes to apoB is another way to decrease levels of lipoproteins in blood. Thus, knowledge of the MTP specific binding site is useful in the development of assays to identify compounds which specifically modulate binding of MTP or MTP/lipid complexes to apoB. Compositions which modulate binding, i.e., increase or decrease binding of MTP or MTP/lipid complexes to the identified MTP specific binding site of apoB are useful in modulating lipoprotein biosynthesis and secretion.

Identification of MTP specific binding site of apoB

Different regions of apoB were expressed as FLAG/apoB chimeras. FLAG (DYKDDDDK) SEQ ID No. 1 is an octapeptide that is commonly used as an epitope tag. cDNAs were transiently transfected into COS cells and conditioned media were used to examine the secretion of FLAG/apoB chimeras using an anti-FLAG monoclonal antibody, M2 (Table 1). Microtiter wells were coated with M2 (3 μg/well) or purified, heterodimeric MTP (1 μg/well). Wells were incubated in triplicate with 100 μl of different conditioned media, washed, and then bound peptides were quantified using polyclonal sheep anti-human apoB antibodies and alkaline phosphate-labeled anti-sheep IgG (Hussain, M. M. et al. 1997. Biochemistry 36:13060-13067). The optical densities were determined at 405 nm.

TABLE 1
Binding of Different apoB Sequences to Immobilized MTP

| apoB | | | | |
|---|---|---|---|---|
| Sequences | Anti-FLAG | | | Binding |
| (amino | M2 | MTP | Ratio | (% of 250- |
| acids) | (O.D.)¹ | (O.D.) | (MTP/M2) | 570) |
| 270-570 | 0.76 | 0.71 (0.15) | 0.93 | 100 |
| | (0.10)² | | | |
| 1-300 | 0.85 (0.02) | 0.03 (0.02) | 0.04 | 4 |
| 1-502 | 0.73 (0.11) | 0.18 (0.07) | 0.25 | 27 |
| No DNA | 0.00 | 0.00 | — | — |

¹O.D. = optical density reading
²Values in parentheses represents the standard deviation.

Thus, FLAG/apoB chimeras containing amino acids 1-300, 270-570, and 1-502 were secreted to a similar extent, which is consistent with earlier metabolic labeling studies (Shelness, G. S. et al. 1994. J. Biol. Chem. 269:9310-9318).

The binding of secreted peptides to immobilized MTP was also studied and compared with their binding to M2 (Table 1). Similar amounts of amino acids 270-570 bound to M2 and MTP (MTP:M2 ratio of 0.93). In contrast, amino acids 1-502 interacted poorly with immobilized MTP with the amount bound to MTP being only 25% of that bound to M2. Thus, when compared to amino acids 270-570, amino acids 1-502 exhibited only 30% of the MTP binding activity. Accordingly, optimum MTP binding occurred with amino acids 270-570 of apoB and truncation of amino acids 502 to 570 resulted in significant (>70%) loss of binding.

Binding interactions were also studied in solution. In these experiments, MTP or M2 was incubated with radiolabeled amino acids 1-300 and 270-570 and complexes were recovered by immunoprecipitation with antibodies to MTP or M2. The amount of amino acids 1-300 co-immunoprecipitated with M2 was six-fold higher than that of amino acids 270-570 indicating that the amount of amino acids 1-300 secreted was much higher. Nonetheless, only amino acids 270-570 was co-immunoprecipitated with MTP. The amount of amino acids 270-570 co-immunoprecipitated with M2 and MTP were similar. In control experiments, anti-mouse IgG or anti-MTP IgG did not co-immunoprecipitate amino acids 1-300 or 270-570. Thus, amino acids 270-570 recognizes both soluble and immobilized MTP.

The specific size of the MTP binding site in amino acids 270-570 was examined. Amino acids were deleted from the C-terminus of amino acids 270-570 with consideration be given to truncating polypeptides at naturally occurring proline residues to avoid disruption of helical structures. Plasmids expressing various truncated forms of the 270-570 amino acid sequence were transiently transfected in COS cells, and the amounts of synthesized and secreted peptides were determined by radiolabeling, immunoprecipitation, and exposure to Phosphorimager screens. The amounts of all of the FLAG/apoB chimeras in cell lysates were similar indicating that they were synthesized to a similar extent by the transfected cells. However, the amounts of secreted chimeras were very different. Amino acids 270-570 was most efficiently secreted by these cells, while amino acids 270-394 was secreted with the least efficiency. The secreted chimeras were also measured by enzyme-linked immunoassay (ELISA) using immobilized M2 (Table 2). The secretion efficiency observed for these peptides was amino acids 270-570>270-509 and 270-430>270-341>270-394, which is consistent with results obtained from radiolabeling and immunoprecipitation.

The binding of these peptides to MTP was also determined (Table 2). Amounts of amino acids 270-570 which bound to M2 and MTP were similar since the ratio between their binding was close to one. This binding is consistent with the data in Table 1. The ratio between the binding of amino acids 270-509 to M2 and MTP was 0.52 indicating that loss of amino acids 510-570 decreased the binding to MTP by ≈60% (Table 2). Truncation of amino acids 270-570 to amino acids 270-430 and amino acids 270-341 resulted in complete loss of binding to MTP. This loss was not due to low levels of the chimeras being secreted because the binding of amino acids 270-430 to M2 was similar to that observed for amino acids 270-509 as determined by the extent of color development. It is believed that removal of amino acids 430-570 resulted in loss of MTP binding. Thus, these studies indicate that an MTP binding site is present within amino acids 430-570 apoB or these amino acids are crucial for MTP binding.

TABLE 2
Effect of C-Terminal Truncations on Binding to Immobilized MTP

| apoB | | | | |
|---|---|---|---|---|
| Sequences | Anti-FLAG | | | Binding |
| (amino | M2 | MTP | Ratio | (% of 270- |
| acids) | (O.D.)¹ | (O.D.) | (MTP/M2) | 570) |
| 270-570 | 0.59 | 0.53 (0.05) | 0.90 | 100 |
| | (0.02)² | | | |
| 270-509 | 0.42 (0.03) | 0.22 (0.02) | 0.52 | 58 |
| 270-430 | 0.40 (0.02) | 0.03 (0.02) | 0.08 | 9 |
| 270-394 | 0.05 (0.02) | 0.00 (0.01) | 0.00 | 0.00 |
| 270-341 | 0.27 (0.03) | 0.00 (0.01) | 0.00 | 0.00 |

¹O.D. = optical density reading
²Values in parentheses represents the standard deviation.

The region of apoB containing amino acids 430-570 was subjected to secondary structure prediction by the Chou-Fasman method using the Wisconsin Sequence Analysis Package of the Genetics Computer Group. This method predicted two α-helices consisting of amino acids 496-508 and 529-541. Helical wheel plots of these peptides indicated that helix₄₉₆₋₅₀₈ is amphiphilic, while helix₅₂₉₋₅₄₁ is not. Both helices contain three positively charged residues. In helix₄₉₆₋₅₀₈, the hydrophilic region contains lys-498, arg-505, and lys-506, whereas helix₅₂₉₋₅₄₁ contained lys-530, arg-531 and arg-540. Lysine and arginine residues are crucial for the binding of MTP to apoB. Thus, it is believed that these two α-helices play an important role in MTP binding to apoB. No other homologous sequences similar to either amino acids 430-570 or the identified helices were identified in the apoB100 molecule.

Importance of MTP binding at this site in apoB

Studies were performed to determine whether apoB/MTP binding was required for secretion of apoB-containing lipoproteins. Several compounds were screened. In the absence of any test compound, 15.47 fmol of low density lipoproteins (LDL)which contain ApoB was bound to immobilized MTP. Two test compounds, AGI-3 and AGI-S17, inhibited 70 to 90% of the binding at 40-50 μM concentrations. In contrast, BMS-200150, an inhibitor of MTP's lipid transfer activity, did not effect LDL/MTP binding. The effect of these same compounds on MTP's lipid transfer activity was also evaluated. BMS-200150 inhibited triglyceride transfer activity by 60% at concentrations in the range of 20 to 50 μM. AGI-3 inhibited only 20-30% of the activity at these same concentrations. In contrast, AGI-S17 did not inhibit the triglyceride transfer activity of MTP. These data indicate that AGI-3 inhibits both LDL/MTP interactions and MTP activity, whereas AGI-S17 inhibits LDL/MTP binding without affecting lipid transfer activity of MTP.

The effect of AGI-S17 on the binding of apoB100 to MTP and its monoclonal antibody 1D1, which recognizes amino acids 474-539 in apoB, was examined. AGI-S17 inhibited the binding of apoB100 to MTP by about 60% (4-50 μM concentrations) but had no effect on the binding of LDL to 1D1. These data indicate that AGI-S17 specifically inhibits the binding of apoB100 to MTP. The effects of AGI-S17 on apoB18 binding to MTP was also tested. Again, the compound was shown to inhibit binding significantly.

The effect of AGI-S17 on apoB secretion in cultured cells was examined. HepG2 cells were incubated with different concentrations of AGI-S17 and the secretion of apoB was determined. At 20-30 μM concentrations, AGI-S17 had no effect on the amount of apoB secreted. However, treatment of cells with 40 μM AGI-S17 resulted in a 48% decrease in the secretion of apoB100.

The effect of AGI-S17 on the binding of amino acids 270-570 to MTP was also examined. AGI-S17 at a 10 μM concentration inhibited 70% of the binding of amino acids 270-570 to immobilized MTP, but did not inhibit binding to immobilized M2.

The effect of lipids on the binding of MTP with apoB was also examined. Phosphatidylcholine (PC) and sphingomyelin increased the interactions by 2 and 1.5 times respectively. In similar studies, PC vesicles at a concentration of 2.4 mM increased the binding of LDL to MTP by 2 to 4 times. As a control, the effect of phospholipids on the binding of LDL to the monoclonal antibody 1D1 and polyclonal antibodies was studied. 1D1 binds to a portion of the MTP specific binding site on apoB. This antibody also recognizes all subclasses of VLDL and LDL, as well as delipidated apoB. PC slightly increased the binding of LDL to 1D1, whereas sphingomyelin had an inhibitory effect (50% inhibition at 2.4 mM). The effect of PC vesicles on the interaction of apoB to polyclonal antibodies was studied by incubating immobilized LDL with the antibodies both in the presence and absence of PC. PC vesicles had no effect on binding.

To study the effect of triglycerides (TG) on apoB/MTP specific binding, PC vesicles or emulsions containing two different concentrations of TG were prepared and their effect on LDL binding to MTP was compared with those of PC vesicles with no TG. PC vesicles enhanced LDL binding in a dose-dependent manner. Inclusion of TG had no additional effect on PC-mediated enhanced binding of LDL to MTP.

The effect of different lipids was studied in more detail using PC-based vesicles (Table 3). Again, PC vesicles increased the binding of LDL to MTP by 3-fold. Addition of sphingomyelin (SPH) or cholesterol to PC vesicles had no additional effect. However, inclusion of negatively charged phospholipids, phosphatidyl inositol (PI) and phosphatidyl serine (PS) in PC vesicles significantly decreased the PC- stimulated binding of LDL to MTP. These results indicate that zwitterionic phospholipids promote, whereas negatively charged phospholipids decrease apoB-MTP interactions.

TABLE 3
Effect of Different Lipids on Interactions Between apoB and MTP

| | | Net | LDL | | |
|---|---|---|---|---|---|
| Vesicles | | Vesicle | Bound | Percent | |
| (mM) | n² | Charge | (fmol) | Inhibition | p-value³ |
| None | 6 | | 16.05⁴ | | |
| | | | (0.98) | | |
| PC (2.4) | 3 | 0.00 | 46.79 | | |
| | | | (2.83) | | |
| PC (4.8) | 3 | 0.00 | 45.20 | | |
| | | | (1.02) | | |
| PC + | 3 | 0.00 | 47.32 | 0.00 | |
| sphingomyelin | | | (0.95) | | |
| (2.4 + 2.4) | | | | | |
| PC + Chol | 3 | 0.00 | 42.03 | 7 | |
| (2.4 + 2.4) | | | (0.37) | | |
| PC + PI | 3 | −1 | 26.72 | 41 | <0.001 |
| (2.4 + 2.4) | | | (0.74) | | |
| PC + PS | 3 | −1 | 33.28 | 26 | <0.001 |
| (2.4 + 2.4) | | | (1.01) | | |

¹Type of vesicle tested, concentration in parentheses.
²n = number of wells used per treatment
³Significant differences evaluated using a Student's t-test
⁴Mean values reported with standard deviation in parentheses

The effect of phospholipids on the kinetic parameters of apoB-MTP binding was examined. It was found that the effect of phospholipid vesicles on the binding of LDL to immobilized MTP was more pronounced at lower concentrations. No significant binding of LDL to MTP could be observed at low concentrations (≦3 nM) in the absence of vesicles. However, the effect of vesicles was significant at ≦12.5 nM of LDL. At higher concentrations the binding of LDL to immobilized MTP in the presence and absence of vesicles was not significantly different. Nonlinear regression analysis revealed that phospholipids decreased the dissociation constant (K_{d}; 23±8 versus 69±6, mean±standard error) by three-fold without significantly affecting the maximum binding capacity (Bₘₐₓ; 13±0.5 versus 10±1.2, mean±standard error). In four other experiments, decreases in K_{d} ranged between 1.5- to 3-fold. The effect of phospholipid vesicles on the binding of ¹²⁵I-MTP to immobilized LDL was also examined. In these experiments, the binding of ¹²⁵I-MTP in the absence of phospholipids did not reach saturation, and thus, K_{d} and Bₘₐₓ values were not determined. Nonetheless, the increase in the binding of ¹²⁵I-MTP was significantly higher (3-fold at 2-8 nM) at lower concentrations than at higher concentrations (2-fold at 17 and 34 nM). Furthermore, at the highest concentration tested (68 nM) the differences in binding were no longer statistically significant, indicating that the effect of PC vesicles was probably on the K_{d} and not on the Bₘₐₓ. Considered together, these results indicated that phospholipid vesicles increase affinity between apoB and MTP without altering the number of binding sites.

The interactions between apoB28 with PC vesicles were also examined. Conditioned medium obtained from McA-RH7777 cells stably transfected with human apoB28 was incubated with lipid vesicles. Preincubation of conditioned medium with PC vesicles resulted in a slight shift of apoB28 peak toward lower density. ApoB28 was separated from the lipid vesicles. Fractions containing apoB28 were pooled, dialyzed and their binding to immobilized MTP examined. The binding of apoB28 preincubated with or without phospholipid vesicles was similar, indicating that interactions between lipids and apoB28 were probably not important for the enhanced interactions between apoB and MTP.

Further experiments were performed to examine MTP interactions with PC vesicles. Studies were done to determine whether MTP binds to PC vesicles by incubating MTP with labeled vesicles and then determining the floatation properties of the vesicles. PC vesicles had a floatation density of plasma LDL and eluted as a single homogeneous peak. In contrast, incubation of vesicles with MTP resulted in a decrease in the vesicle peak and in the appearance of a hump at the trailing end of the vesicle peak. These data indicate that MTP changed the floatation properties of some of the PC vesicles, most likely by interaction with the vesicles. To determine whether MTP interacted with PC vesicles, ¹²⁵I-MTP was incubated with or without PC vesicles and subjected to density gradient ultracentrifugation. MTP, incubated without lipid vesicles, was recovered in the bottom fractions corresponding to a density >1.21 g/ml. This was designated peak A (fractions 21-24). In contrast, MTP incubated with vesicles was distributed in two fractions. One fraction was recovered as lipid-poor MTP (designated peak B, fractions 21-24), similar to that observed for MTP alone (peak A). This fraction may represent MTP with 1-2 mol of lipid molecules. MTP associated with lipids (≈50%) was also recovered (designated peak C, fractions 3-9). The profile of MTP associated with vesicles was similar to that observed for vesicles incubated with unlabeled MTP, indicating that MTP interacted with all of the vesicles. Further, vesicles contained various amounts of MTP, leading to two different populations of vesicles containing different amounts of MTP. Thus, these data indicate that MTP forms stable complexes with lipid vesicles.

Binding of MTP/lipid complexes to apoB

To study the binding of lipid associated MTP with LDL, peaks A, B, and C were pooled, dialyzed and then used to study binding to immobilized LDL. The binding of lipid-poor MTP (peak B), which might have acquired few lipid molecules during its incubation with lipid vesicles, to LDL was increased (70%, P<0.005) compared to the binding of MTP not incubated with lipids (peak A). More importantly, the amount of MTP/lipid complexes (peak C) bound to LDL was three-fold higher than when MTP was used alone. These studies indicate that lipid-associated MTP binds better to LDL as compared to lipid-poor and lipid-free MTP. It is believed that MTP forms stable complexes with phospholipid vesicles and these MTP/lipid complexes have higher affinity for apoB.

The binding of apoA-I and apoA-I/lipid complexes to immobilized LDL was examined. ApoA-I incubated with or without lipid vesicles was subjected to ultracentrifugation and apoA-I/lipid complexes were isolated. Binding of apoA-I and apoA-I/lipid complexes to immobilized LDL was then examined. In contrast to the observations with MTP, binding of apoA-I/lipid complexes to LDL were significantly less than the binding of free apoA-I. These results indicate that increased binding of MTP/lipid complexes was not due to interactions between lipids, and that the increased interactions between immobilized LDL and MTP/lipid complexes were dependent on the presence of MTP.

Presence of MTP/lipid complexes in the lumen of microsomes isolated from HepG2 cells

The possibility that some of the MTP may exist bound to lipids in the lumen of the endoplasmic reticulum and float at a density<1.21 g/ml was evaluated. Consideration was given to the possibility that MTP would float if associated with apoB-containing lipoprotein. Thus, attempts were made to identify lipid-bound MTP that was not associated with apoB. First, lumenal proteins from microsomes of HepG2 cells were subjected to density gradient ultracentrifugation, different fractions were collected, and apoB was immunoprecipitated from these fractions under non-denaturing conditions. Proteins in the immunoprecipitates and supernatants were distributed in half, separated on gels, transferred to nitrocellulose, and probed with either anti-apoB or anti-MTP antibodies. Western blotting with anti-apoB antibodies revealed that apoB was present in the immunoprecipitates but not in the supernatants indicating that all of the lumenal apoB was precipitated with antibodies. Full length apoB100 was distributed in various density fractions and probably represented various intermediates of lipoprotein biosynthesis. The distribution of MTP in different fractions immunoprecipitated or not with apoB was then determined. MTP was present in all the fractions immunoprecipitated with anti-apoB antibodies indicating its association with apoB in various intracellular lipoproteins. In addition, MTP was also present in proteins that were not immunoprecipitated with anti-apoB and is believed to represent free MTP. The free MTP was present in different density fractions corresponding to flotation densities of 1.02-1.063 and<1.21 g/ml indicating that various amounts of MTP were present associated or not with lipid vesicles. Thus, these studies indicate that, at steady state conditions, MTP is present in three forms, free; bound to apoB, and associated with lipid vesicles.

Accordingly, three distinct means for modulating lipid and lipoprotein secretion have now been identified. The present invention relates to methods of identifying compounds which alter interaction of MTP or MTP/lipid complexes with apoB and the interaction of MTP with lipids. In a preferred embodiment, the present invention provides a method for identifying compounds which inhibit binding of MTP to an MTP specific binding site of apoB. In this method, compounds are screened in an assay which measures binding of MTP to a MTP specific binding site on apolipoprotein B. Examples of such assays include, but are not limited to, measurement of binding of an apoB containing lipoprotein such as LDL or a fragment of apoB containing the MTP specific binding site to immobilized MTP in the presence and absence of a test compound and measurement of binding of MTP to an immobilized apoB containing lipoprotein such as LDL or an immobilized fragment of apoB containing the MTP specific binding site in the presence and absence of a test compound. Methods of quantitating LDL bound to MTP by ELISA have been described by Hussain et al. 1995 Arterioscl. Throrb. Vasc. Biol. 15:485-494 and Bakillah et al. 1997 Lipids 32:1113-1118. Compounds identified by this method which decrease binding will decrease apoB secretion, thereby decreasing secretion of lipids. A method is also provided for identifying compounds that inhibit binding of MTP/lipid complexes to an MTP specific binding site of apoB. In this method, compounds are screened in an assay which measures binding of MTP/lipid complexes to a MTP specific binding site on apolipoprotein B. Examples of such assays include, but are not limited to, measurement of binding of MTP/lipid complexes to immobilized apoB containing lipoproteins such as LDL or a fragment of apoB containing the MTP specific binding site in the presence and absence of test compounds and measurement of binding of apoB containing lipoproteins such as LDL or a fragment of apoB containing the MTP specific binding site to immobilized MTP in the presence of phosphatidylcholine vesicles and in the presence and absence of test compounds. Compounds identified by this method as inhibitors of binding will decrease secretion of larger lipoproteins, thereby also decreasing lipid biosynthesis. In addition, methods are provided for identifying compounds that modulate the association of MTP with lipids. In this method compounds are screened in an assay which measures binding of microsomal triglyceride transfer protein to lipid complexes. In one embodiment, the assay involves incubation of purified MTP with phosphatidylcholine vesicles in the presence and absence of test compounds followed by ultracentrifugation of the incubation mixture and determination of the distribution of MTP in different fractions via Western blotting. Compounds identified as inhibitors of MTP/lipid complex formation are expected to inhibit lipoprotein and lipid secretion. Thus, by using the methods of the present invention, novel compositions can be identified that can be used to lower levels of plasma lipoproteins.

The following nonlimiting examples are provided to illustrate the claimed invention.

### EXAMPLES

Example 1:

Preparation of Cells in Culture

HepG2 and Cos-7 cells were obtained from the American Type Culture Collection (Rockville, Md.) and cultured in minimal essential medium of Dulbecco's modified minimal essential medium, respectively, containing 10% fetal bovine serum and 1% antibiotic-antimycotic. McA-RH7777 cells stably transfected with apoB28 and apoB18 were grown in Dulbecco's modified medium containing 10% fetal bovine serum and 1% antibiotic-antimycotic (Hussain, M. M. et al. 1995. Arterioscler. Thromb. Vasc. Biol. 15:485-494).

Example 2:

Preparation of Lipid Vesicles

Required amounts of lipids in chloroform were added to a glass vial and the solvent was evaporated under nitrogen gas. A calculated amount of the buffer (15 mM Tris-HCl, 1 mM EDTA, 0.02% sodium azide, pH 7.4) was added, and the contents were sonicated (setting 5, output 50-60%) on ice until the solution was mostly clear. The vesicles were then centrifuged at 3500 rpm for 15 minutes, 4° C., and the supernatant used to assay the effect of vesicles on binding.

Example 3:

Expression of FLAG/apoB Chimeras in COS Cells

To identify a putative MTP binding site in apoB, several FLAG/apoB chimeras were constructed. Various apoB DNA sequences were amplified by the polymerase chain reaction and cloned into NotI and ApaI site of the pCMV/FLAG expression vector (Huang, X. F. and G. S. Shelness 1997 J. Biol. Chem. 272:31872-31876; Shelness, G. S. et al. 1994 J. Biol. Chem. 269:9310-9318) . The expression plasmids contained the CMV immediate early promoter, a 30 amino acid signal sequence from bovine preprolactin, the FLAG octapeptide, and 33 amino acid of mature bovine prolactin, followed by the pertinent apoB sequences. The vectors were transiently expressed in COS cells using diethyl aminoethyl-dextran (Huang, X. F. and G. S. Shelness 1997 J. Biol. Chem. 272:31872-31876; Shelness, G.S. et al. 1994 J. Biol. Chem. 269:9310-9318; Sambrook, J. et al. 1989 Molecular Cloning: A Laboratory Manual, Cold Spring Harbor Press: New York). Conditioned media were used to measure the secreted chimeras by virtue of their binding to M2 and were simultaneously used to study MTP binding.

Example 4:

Binding of FLAG/apoB Chimeras to Immobilized MTP and M2

M2 (3 μg/well) or MTP (1 μg/well) were immobilized (2 hours at 37° C. or 18 hours at 4° C.) in microtiter plates, washed (3 times) , and incubated (30 minutes, 37° C.) with PBS-Tween. After three washes with PBS-Tween, the microtiter wells were incubated with conditioned media from transfected cells for 2 hours at 37° C. in triplicate. For controls, triplicate wells were incubated with non-conditioned media or PBS-Tween. Wells were then washed with PBS-Tween, incubated with sheep anti-human apoB polyclonal antibodies (1:2000 dilutions) for 1 hour at 37° C., washed, incubated (1 hour, 37° C.) with alkaline phosphatase-labeled anti-sheep IgG, washed, and incubated with p-nitrophenyl phosphate (1 mg/mL in 10 mM ethanolamine, 0.5 MM MgCl₂, pH 9.5) for various times. The absorbance at 405 nm was determined using a Microplate Reader (Dynatech Labs, Chantilly, Va.). The highest optical densities from control wells were subtracted to determine the specific binding to M2 and MTP. The values in M2 coated wells provided evidence for the secretion of different chimeras, whereas the values in MTP coated wells represented binding to MTP.

Example 5:

Binding of FLAG/apoB Chimeras to Soluble MTP and M2

Transfected COS cells were radiolabeled with [ ³⁵S] protein labeling mixture (NEN/Du Pont) and conditioned media (1 ml) were incubated overnight with purified heterodimeric MTP (1 μg/ml), followed by rabbit anti-bovine MTP (5 μl) and anti-rabbit IgG (5 μl). In parallel, conditioned media (1 ml) were incubated with M2 (15 μg/ml), and anti-mouse IgG. Immune complexes were recovered using protein G-Sepharose (Pharmacia) , washed, separated on polyacrylamide gels, and autoradiographed using PhosphorImager 445SI (Molecular Dynamics, Sunnyvale, Calif.).

Example 6:

Effect of Agents on MTP Binding

ELISA plates coated in triplicate with purified heterodimeric MTP (1 μg/well) were incubated with human LDL (1 μg/well) in the presence of indicated concentrations of various compounds. Microtiter wells were washed three times with PBS-Tween and apoB bound was quantitated by a sandwich ELISA (Hussain, M. M. et al. 1995 Arterioscl. Thronb. Vasc. Biol. 15:485-494). In parallel, a standard curve for apoB was generated by coating wells with a monoclonal antibody, 1D1, and incubating with different concentrations of LDL (0-14 ng/well) as previously described (Hussain, M. M. et al. 1995 Arterioscl. Thromb . Vasc. Biol. 15:485-494).

Example 7:

Modulation of MTP Binding by Lipids

LDL (20 nM) was incubated with immobilized MTP (1 μg/ml) in the presence and absence of different concentrations of PC or sphingomyelin vesicles for 2 hours at 37° C. and the amount of LDL bound quantitated. The amount of LDL bound in the absence of lipid vesicles was used as 100%. Parallel experiments were also performed wherein LDL (78 pM) was incubated with immobilized monoclonal antibody, 1D1, instead of MTP. LDL (20 nM) was also incubated with immobilized MTP in the presence of different concentrations of PC, PC/TG (molar ratio 400:1) vesicles, or PC/TG dispersions (molar ratio 1:1) for 2 hours at 37° C., and the amount of apoB bound was quantitated by ELISA.

What is claimed:

1. A method of identifying compounds which modulate lipid secretion comprising screening compounds in an assay which measures binding of microsomal triglyceride transfer protein to a microsomal triglyceride transfer protein specific binding site on apolipoprotein B.

2. A method of identifying compounds which modulate lipid secretion comprising screening compositions in an assay which measures binding of microsomal triglyceride transfer protein-lipid complexes to a microsomal triglyceride transfer protein-lipid complex binding site on apolipoprotein B.

3. A method of identifying compounds which modulate lipid and lipoprotein secretion comprising screening of compounds in an assay which measures binding of microsomal triglyceride transfer protein to lipid complexes.


SEQUENCE LISTING (1 sequence)

SEQ ID NO: 1 - protein, 8 aa, Artificial Sequence
  Description of Artificial SequenceSynthetic
DYKDDDDK